(19) United States
(12) Patent Application Publication
(10) Pub. No.: US 2008/0142007 A1
(43) Pub. Date: Jun. 19, 2008

(54) Medicament dispenser for an inhaler

(75) Inventor: Derek Fenlon, Co. Waterford (IE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Norton Healthcare Ltd, London (GB)

(21) Appl. No.: 11/639,643

(22) Filed: Dec. 15, 2006

(51) Int. Cl. A61M 15/00

(52) U.S. Cl. 128/203.15

(57) ABSTRACT

An inhaler for administering a predetermined dose of a medicament. The inhaler includes a screw-threaded mechanism which is operable to dispense the predetermined dose for inhalation. The inhaler can include a rotatable threaded member mounted within a casing. The inhaler can also include an internally threaded sleeve mounted on the rotatable threaded member. The sleeve can be moved linearly by rotation of the threaded member. Medicament can be provided in a tray of the sleeve, and can be aligned with a mouth piece of the inhaler for inhalation by the linear movement of the sleeve.

### BACKGROUND OF THE INVENTION

This invention relates to an inhaler. In particular, this invention relates to an inhaler for dispensing a predetermined dose of a medicament.

Inhalers are commonly known for use in administering a medicament. The medicament may be provided in, for example, a powdered form and can be drawn out of a mouthpiece of the device and inhaled by a user.

Some inhalers are disposable in the sense that they are designed to be thrown away once the medicament therein has been exhausted. Other inhalers are reusable, and can be replenished by receiving replaceable cartridges in which medicament is stored.

Some known inhaler designs include an overly complex construction having a large number of separate parts which must be assembled. This increases manufacturing costs, which is particularly disadvantageous in the case of disposable inhalers. A complex design may also result in an inhaler which is not easy to use.

It will be appreciated that an inhaler must also be capable of the administration of a reliable dose of medicament. Where multiple uses of the inhaler are envisaged, the inhaler must be able repeatedly to administer a predetermined dose.

Examples of known inhalers are described in WO 1997/02061 and EP 0528764. WO/1997/02061 describes an inhaler for administering medicaments from blister packs. Blister packs are well known in the field of medicament administration as a means for storing a predetermined dosage. The inhaler described in WO 1997/02061 includes a housing within which a blister strip can be inserted. A number of plungers are provided in the housing for pressing out the individual cavities of the blister strip, to release the medicament stored therein for inhalation.

EP 0 528 764 describes an inhaler for administration of powdery substances. The inhaler includes an elongate body having a magazine passage that extends therethrough. An elongate magazine is provided, which has capsule chambers spaced along its length. The elongate magazine is slideable stepwise in and along the magazine passage to align the capsule chambers with an air inlet, an air outlet and capsule cutting means.

### SUMMARY OF THE INVENTION

Particular and preferred aspects of the invention are set out in the accompanying independent and dependent claims. Combinations of features from the dependent claims may be combined with features of the independent claims as appropriate and not merely as explicitly set out in the claims.

According to an aspect of the invention, there can be provided an inhaler for administering a predetermined dose of a medicament. The inhaler includes a screw-threaded mechanism operable to dispense the predetermined dose for inhalation.

The screw threaded mechanism provides a robust and reliable means for dispensing doses of medicament to be inhaled. The screw threaded mechanism also allows for precise positioning of components of the inhaler, thereby allowing controlled dispensing of doses of medicament for inhalation. Furthermore, the screw threaded mechanism can be constructed using a minimum of parts, thereby minimizing manufacturing costs.

The inhaler can include a rotatable threaded member mounted within a casing. An internally threaded sleeve can be mounted on the rotatable threaded member such that rotation of the threaded member within the casing leads to linear movement of the sleeve. This linear movement can be employed to align medicament with means such as a mouth piece of the inhaler.

The sleeve can include a tray for storing doses of medicament. The tray can be integral with the sleeve. The tray can be replaceable. For example, the tray or the sleeve with the tray in situ can be removed from the casing and replaced to replenish the medicament in the inhaler. The tray may include a cover that can be broken or removed (e.g., peeled back) as the sleeve moves linearly within the casing, thereby exposing a predetermined portion of the medicament in the tray for inhalation.

The inhaler can have a pen-like construction. The casing can be elongate and have a longitudinal axis, and the rotatable threaded member can be substantially coaxially mounted within the casing along the longitudinal axis. This constitutes a particularly compact construction. A mouthpiece can be laterally positioned on a side of the elongate casing, as can any air inlets.

The rotatable threaded member can include a handle portion which protrudes from the casing to allow rotation of the threaded member by a user.

The pitch of the thread of the threaded member can be chosen to provide for precise adjustment of the position of the sleeve within the casing. For example, the thread of the threaded member can have a pitch of between about 1 and about 10 threads/cm, preferably between about 2 and about 5 threads/cm.

The inhaler can include means for indicating to a user information representative of the position of the sleeve within the casing. This can allow the user to determine the status of the medicament within the inhaler, and in particular, determine the correct dosage to be inhaled and the amount of medicament remaining in the inhaler. For example, a window can be provided in the casing to allow dosage indications provided on the threaded member to be viewed.

The symbols can be located on a surface of the rotatable threaded member. In this way, rotation of the threaded member can cause alignment of the symbols with respect to the window to indicate to the user the information representative of the position of the sleeve within the casing. These symbols can be located on teeth of the rotatable threaded member.

The symbols can also be located on a surface of the sleeve. In this way, linear movement of the sleeve within the casing can cause alignment of the symbols with respect to the window to indicate to the user the information representative of the position of the sleeve within the casing.

Means can be provided for locking the threaded member to inhibit inadvertent rotation of the threaded member within the casing.

Means can be provided for allowing stepwise rotation of the threaded member, whereby the threaded member can be controllably rotated by a predetermined amount. The predetermined amount can, for example, correspond to the dispensing of a predetermined dosage of the medicament.

The threaded member may only be rotatable in a first direction within the casing. This can minimize the risk of confusion regarding the dosage remaining within the inhaler. For example, a ratchet mechanism can be provided for inhibiting rotation of the threaded member in a direction opposite to the first direction within the casing.

A lock-out mechanism can be provided for preventing removal of the threaded member from the casing after the

### BRIEF DESCRIPTION OF THE DRAWINGS

For a better understanding of the invention and to show how the same may be carried into effect, reference is now made by way of example only to the accompanying drawings in which like reference signs relate to like elements and in which:

FIG. 1 is a front perspective view of an inhaler according to an embodiment of the invention.

FIG. 2 is a rear perspective view of the inhaler shown in FIG. 1.

FIG. 3 is an exploded view of the inhaler shown in FIGS. 1 and 2.

FIG. 4 is a longitudinal cross-sectional view of the inhaler shown in FIGS. 1 and 2.

While the invention is susceptible to various modifications and alternative forms, specific embodiments are shown by way of example in the drawings and are herein described in detail. It should be understood, however, that the drawings and the detailed description thereof are not intended to limit the invention to the particular form disclosed, but on the contrary, the invention is to cover all modifications, equivalents and alternatives falling within the spirit and scope of the present invention as defined by the appended claims.

### DETAILED DESCRIPTION

Particular embodiments will now be described by way of example only in the following with reference to the accompanying drawings.

According to an embodiment of the invention, there can be provided an inhaler for administering a predetermined dose of a medicament.

A wide range of medicaments can be administered using an inhaler according to an embodiment of this invention. Examples of medicaments which can be used include those which are provided in the form of a powder. For example, the powder may include a lactose base to which particles of the medicament are attached. An embodiment of this invention can be used to administer medicaments such as vaccines. An example vaccine is influenza vaccine H5N1 or HIV. Although powders are mentioned, it should be understood that liquids that volatilize easily, or gases may also be administered using the inhaler of the present invention.

Embodiments of this invention can allow for administration of a single dose of the medicament. Alternatively, a plurality of doses of the medicament can be provided within the inhaler for multiple uses. An inhaler of the kind described herein can be used to administer a medicament either orally or nasally.

Inhalers of the kind described herein can be disposable, in that they can be designed with the intention of administering a limited number of doses of medicament before being exhausted. After exhaustion of the medicament in the inhaler, the inhaler can be disposed of. Alternatively, the inhaler can be replenishable. For example, a cartridge containing medicaments can be loaded into the inhaler when a previous cartridge has been exhausted.

The inhaler includes a screw-threaded mechanism that is operable to dispense a predetermined dosage of a medicament for inhalation. As used herein, the term “dispense” refers to the preparation of a dosage of a medicament within the inhaler such that it can be inhaled. This may involve bringing the medicament in the inhaler into an inhaling position. Dispensing may also include, for example, unsealing a container storing the medicament so that it can be inhaled.

Typically, the medicament can be stored in the inhaler in, for example, a sealed cartridge, such that it cannot be inadvertently lost through, for example, a mouthpiece of the inhaler or indeed contaminated by foreign agents entering the inhaler. By dispensing the medicament dose prior to inhalation, the medicament can be positioned such that it can be passed out through the mouthpiece.

The provision of a screw-threaded mechanism that is operable to perform the action for dispensing the dose of medicament allows for precise control of the components in the inhaler. For example, the screw-threaded mechanism can allow for precise and controlled positioning of the medicament within the inhaler. This can, for example, ensure that the correct dosage of medicament is indeed dispensed. The screw-threaded mechanism may also provide for precise control in an unsealing action, so that only a desired portion (e.g., blister) within the inhaler is unsealed.

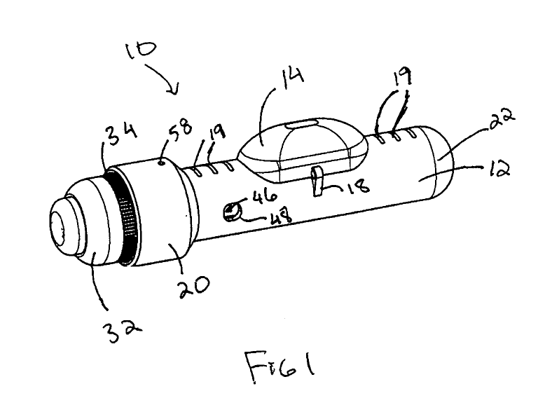

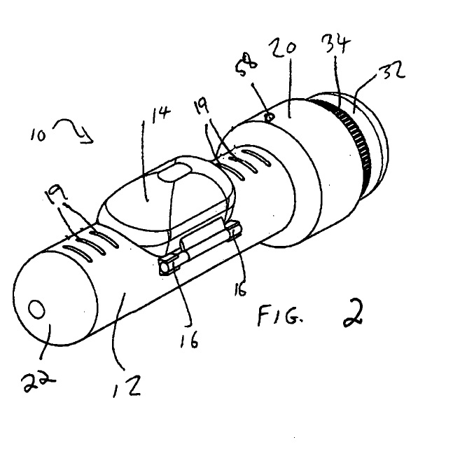

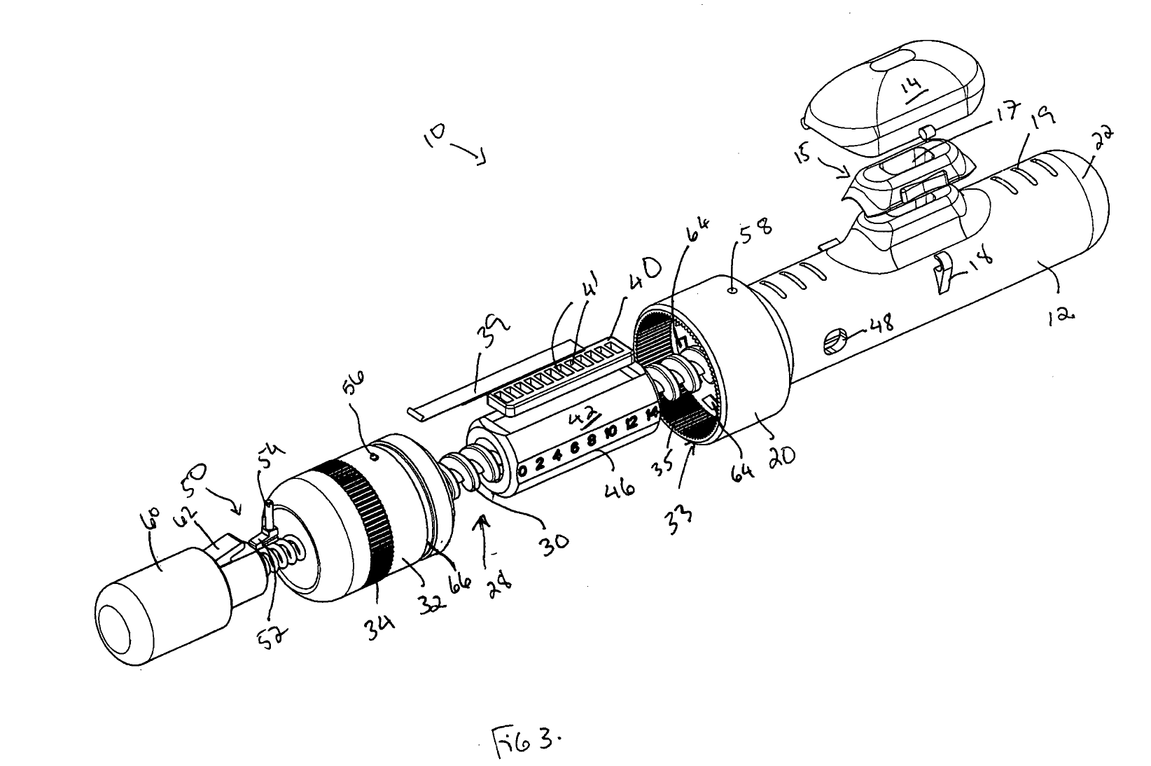

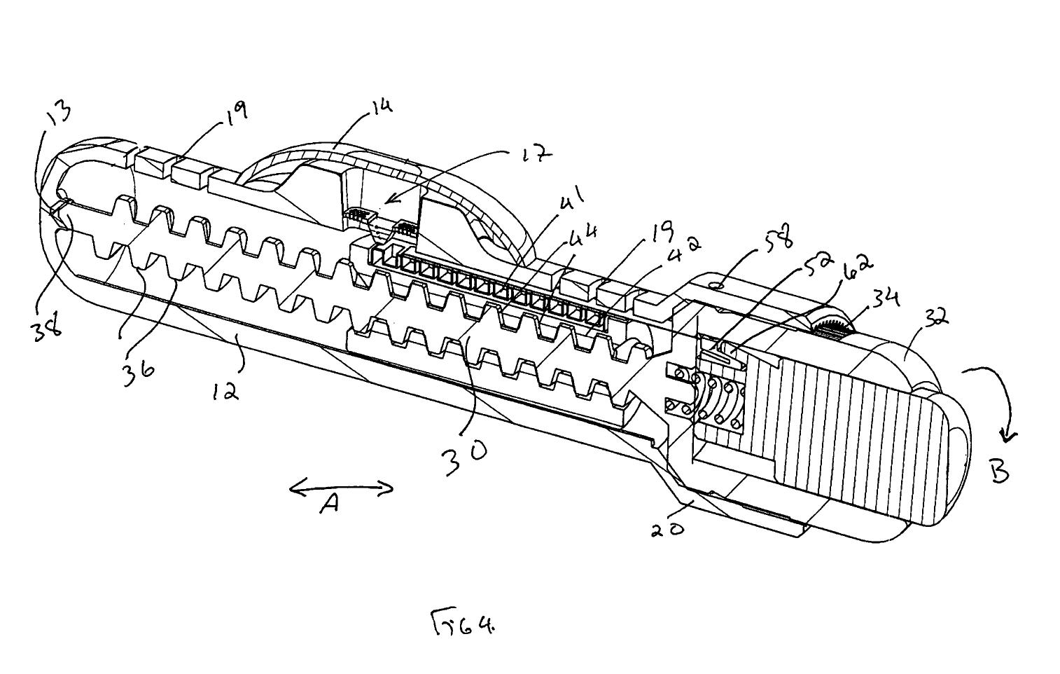

An example of an inhaler according to an embodiment of this invention will now be described in relation to FIGS. 1 to 4. FIGS. 1 and 2 generally show the external features of the inhaler 10. The internal features of the inhaler are discussed in relation to FIGS. 3 and 4.

With reference to FIGS. 1 to 2, an inhaler 10 according to an embodiment of this invention can include a casing 12 which houses a number of internal components. The casing 12 can be constructed from any suitable material such as a metal or a polymer material. Use of a polymer material is advantageous in that it affords a cheap and easy way to manufacture its construction. These considerations are particularly important in the case of an inhaler which is intended to be disposable after a number of uses.

In the present example, the casing 12 of the inhaler 10 is generally cylindrical in shape and has a rounded nose portion 22. It will be understood that the casing 12 ran take other forms and need not be generally cylindrical and/or elongate as shown in the example of FIGS. 1 to 4. For example, the inhaler could be generally box-shaped or could be elongate with, for example, a square, oval, hexagonal or other cross-sectional configuration.

The inhaler 10 can also include an aperture 17 through which a dispensed dosage of a medicament can be inhaled. In the present example, the aperture 17 is formed in a mouthpiece 15, which itself is positioned laterally along a side of the elongate casing 12 of the inhaler 10. The casing 12 in this example also includes a flanged portion 20 which is open ended for receiving a handle 32 of the inhaler 10. As will be described below, the handle 32 in this example is rotatable in order to operate the screw-threaded mechanism 28 for dispensing a dose of medicament stored within the inhaler 10. In order to assist in the rotation of the handle 32, the handle 32 can be provided with, for example, a profiled surface 34 for improved grip.

The mouthpiece 15 of the inhaler 10 can be provided with means for sealing the aperture 17. In the present example, the mouthpiece 15 is provided with a hinged lid 14. As shown in FIGS. 1 and 2, the lid 14 is provided with a number of hinges 16 and a catch 18. The catch 18 can be used to prevent inadvertent opening of the lid 14 when the inhaler is not in use. In one embodiment, the lid can also allow access for the installation/removal and/or replacement of the cartridge for replenishing the medicament within the inhaler.

The inhaler 10 can also include a number of air inlets 19, which allow air to enter the interior of the casing 12 during inhalation of a medicament. In the present example, these inlets 19 take the form of slits which are provided along the length of the casing 12. It will be appreciated that the inlets 19 could take other forms and could be provided in other positions as long as they are suitably positioned to allow appropriate air flow within the casing 12.

As shown in FIGS. 3 and 4, in the present embodiment, the screw-threaded mechanism 28 of the inhaler includes a rotatable threaded member 30 that is mounted within the casing 12. The threaded member 30 is linked to (for example, integrally formed with) the handle 32, whereby rotation of the handle 32 as represented by the arrow labelled “B” in FIG. 4 leads to corresponding rotation of the threaded member 30. Screw-threaded mechanism 28 is held in place in the casing 12 by the mating-engagement of a series of clips 64 disposed circumferentially on the inside of the flanged portion 20 within a groove 66 formed around the circumference of handle 32. By exerting a sufficient pulling force on handle 32, clips 64 can be disengaged from grove 66 to release screw-threaded mechanism 28 for removal from casing 12.

The threaded member 30 in this example has a nose portion 38, at the closed end of the casing 12, which is received in a corresponding recessed portion 13 of the casing 12 to ensure that the threaded member 30 remains substantially co-axial with the casing 12. As described above with respect to the casing 12, the threaded member 30 can be constructed from, for example, a metal or a polymer material.

As shown in FIGS. 3 and 4, the screw-threaded mechanism 28 in this example also includes a sleeve 42, which is mounted on the threaded member 30. The sleeve 42 is internally threaded with threads 44 that correspond to the threads 36 of the threaded member 30. It should be noted that while in the present example the sleeve 42 is annular and encloses the threaded member 30, in other examples the sleeve 42 may only partially encircle the threaded member 30 while still engaging the threads 36 thereof.

On rotation of the threaded member 30 by rotation of the handle portion 32 as represented by the arrow labeled “B,” it can be seen from FIGS. 3 and 4 that the threads 36 of the member 30 and the corresponding threads 44 of the sleeve 42 cooperate to produce linear movement of the sleeve 42 along the threaded member 30 as indicated by the arrow labeled “A.” This linear movement can be used to move a dosage of a medicament stored within the inhaler 10 into an inhaling position. In the present example, this linear movement can be used to position the medicament for inhalation through the aperture 17 of the mouthpiece 15.

In the present example, a plurality of doses of a medicament are stored on the sleeve 42 for dispensing. In particular, the sleeve 42 in this example is provided with a tray 40 within which the medicament is stored. The tray 40 can include a plurality of medicament storage sections 41, each of which is dimensioned to be able to contain a single dosage of the medicament to be administered. From FIGS. 3 and 4 it can be seen that linear motion of the sleeve 42 and the tray 40 can expose one or more of these medicament storage sections 41 to the opening 17 of the mouthpiece 15 for inhalation.

In accordance with an embodiment of the invention, the medicament within the inhaler 10 can be replenished. In the present example, this can be achieved by withdrawing the handle portion 32, the threaded member 30, and the associated sleeve 42 through the open end of the flanged portion 20 of the casing 12. Once withdrawn, the sleeve 42 can be unscrewed from the threaded member 30 and replaced with another sleeve 42 upon which medicament is stored. Alternatively, the tray 40 of the sleeve 42 can be detachable from the sleeve 42. In such case, depleted tray 40 can be detached from sleeve 42 and a new tray 40 containing medicament can be inserted in its place, thereby replenishing the medicament without having to unscrew the sleeve 42 from the threaded member 30. In yet a further example, medicament can simply be placed in the depleted tray 40. After replenishment of the medicament in the sleeve 42, the sleeve 42 can be screwed back on to the threaded member 30 (if necessary) and the threaded member 30 can be reinserted through the open end of the flanged portion 20 of the casing 12.

In some examples, the tray 40 can take the form of a blister pack of the kind which is well known in the art. The tray 40 can be provided with a cover 39 for sealing the storage sections 41 thereof. This cover 39 can be incrementally removed or pierced as the tray 40 moves along the length of the casing 12 to be dispensed for inhalation through the mouthpiece 15. For example, a sharp member such as a small blade or barb may extend beneath the mouth piece 15 toward the tray 40. The blade or barb may be biased toward the tray 40. In this way, the blade or barb can pierce the cover 39 which seals the storage sections 41 of the tray 40 as the storage section 41 appears beneath the aperture 17 of the mouthpiece 15. In the embodiment illustrated, cover 39 may be peeled away to release to release the medicament.

As described above, the use of a screw-threaded mechanism 28 allows for precise movement of a medicament stored within the inhaler 10 and therefore constitutes an advantageous means for dispensing medicament in the inhaler 10 prior to inhalation. With reference to FIGS. 3 and 4, it will be noted that by varying the pitch of the threads 36 of the threaded member 30, the amount of linear movement of the sleeve 42 caused by rotation of the handle 32 and threaded member 30 can be controlled. A finer pitch for the threads 36 reduces the amount of linear movement experienced by the sleeve 42 upon rotation of the handle 32 and conversely, a wider pitch for the threads 36 increases the linear movement of the sleeve 42 for the given amount of rotation of the handle 32. The pitch range is preferably between about 1 and 10 threads/cm, and more preferably between about 2 and 5 threads/cm.

The rotation of the handle 32 and threaded member 30 can also be controlled or constrained in other ways. For example, means can be provided for ensuring that the handle 32, and therefore the threaded member 30, can only be rotated in one direction. This would ensure that the sleeve 42 can only be moved linearly along the length of the casing 12 in a single direction. The purpose of such a feature would be to minimize the risk of an incorrect dose being dispensed within the inhaler, or to minimize doubt as to the amount of medicament which remains within the inhaler 10. In use, the sleeve 42 could be installed at a first extreme end of the elongate casing 12 and then moved in a single direction toward the other extreme end of the casing 12. Since the sleeve 42 cannot be moved in the opposite direction, the situation where, for example, already used storage sections are mistakenly aligned with the mouthpiece 15 for inhalation can be avoided.

In the present example, the means for preventing rotation of the handle 32 in more than one direction within the casing 12 includes a ratchet mechanism 33. In this mechanism 33, ratchet teeth 35 are provided on the interior surface of the flanged portion 20 of the casing 12 and corresponding ratchet teeth 34 are provided on an exterior surface of the handle 32. These ratchet teeth 34 and 35 are so oriented as to only allow rotation of the handle 32 within the flanged portion 20 in a single direction. It will be understood that the ratchet mechanism 33 could be provided elsewhere in the inhaler 10, and need not be provided at the flanged portion 20.

The inhaler 10 can also be provided with a mechanism for locking the threaded member 30 within the casing 12 to pr-event rotation when the inhaler 10 is not being used. In one example, this mechanism could include a catch mechanism 50 which includes a spring 52, having a pin 54 which passes through an aperture 56 in, for example, the handle 32 and into an opening 58 in the flanged portion 20 of the casing 12. Insertion of the pin 54 through the handle 32 and into the aperture 58 would prevent rotation of the handle 32 relative to the flanged portion 20 and thereby lock the screw-threaded mechanism 28, preventing inadvertent dispensing when the inhaler is not being used. Depressing button 60 in the end of handle 32 will cause tongue 62 to compress spring 52, thereby releasing handle 32 for rotation relative to flanged portion 20. Alternatively, longitudinal movement of handle 32 relative to casing 12 may move screw-threaded mechanism 28 between locked and released positions. Other mechanisms for preventing inadvertent rotation of handle 32 will be readily apparent to those skilled in the art and are contemplated herein.

In a further example, the rotation of the threaded member 30 within the casing 12 of the inhaler 10 can be configured to take place in a step-wise manner. The step-wise rotation of, for example, the handle 32 of the inhaler 10 could correspond to the dispensing of a dose of medicament for inhalation. In this way, the user of the inhaler 10 could verify to him or herself that a correct dosage of medicament has been dispensed prior to inhalation.

The step-wise rotation of the threaded member 30 within the casing 12 could be provided by, for example, the ratchet mechanism 33 described above. As the handle 32 rotates within the flanged portion 20, the ratchet teeth 34 and 35 will tend to provide a small and varying amount of resistance to this rotation. As each tooth of the mechanism 33 in, for example, the handle 32 overcomes a corresponding tooth in the flanged portion 20, the resistance to rotation of the handle 32 will slightly decrease and this will be notable by the user. As each tooth passes a corresponding tooth, there also may be produced an audible clicking sound. These indications would both serve to allow the user to determine that rotation of the threaded member 30 and linear movement of the sleeve 42 and tray 40 has occurred. Depending on the thread pitch of threaded member 30, a single rotation (e.g., 360°), numerous rotations or a partial rotation of the threaded member 30 may be needed to advance sleeve 42 and tray 40 by a sufficient amount to dispense the next dose of medicament. Thus, noting the number of clicks that have occurred would allow a user to confirm that a single dosage of medicament has been dispensed for inhalation.

In another example, protrusions such as dimples could be provided on an inner surface of the flanged portion 20 and corresponding recesses could be provided on an outer surface of the handle 32 (it will be appreciated that this arrangement could be essentially reversed). Rotation of the handle 32 within the flange portion 20 would again be step-wise in the sense that the dimples in the flanged portion 20 would engage with the corresponding recesses in the handle 32 in a step-wise manner, thereby allowing the user to determine the number of steps that have been made by the screw-threaded mechanism 28 as described above.

In a further example of the invention, a lock-out mechanism can be provided for the inhaler 10. This lock out mechanism can serve the purpose of preventing the threaded member 30 from being removed from the casing 12, especially after the medicament within the inhaler 10 has been used. For example, it may be desirable to prevent residual medicament, remaining in the inhaler 10 after use, from being accessible. For example, this would prevent children from being able to access the medicament within the inhaler.

A snap feature may be incorporated into the ratchet mechanism so that the connection between the handle 32 and the screw threaded mechanism 28 is disconnected by the user turning the handle once the course of the medicament is completed. The snap feature may break the attachment between the handle 32 and the threaded member 30.

In accordance with a further embodiment of the invention, means can be provided in the inhaler 10 for indicating to a user information regarding the position of the sleeve 42 within the casing 12. This information would in turn allow the user to determine the status of the medicament within the inhaler 10 and, in particular, how much medicament is remaining and whether an additional dose of medicament has been dispensed through the rotation of the handle 32 by the user. These means can take the form of, for example, a counter or means that provide some other kind of indication to the user, such as a color coded indication (e.g., traffic lights).

The information can be encoded in a set of symbols 46 which are provided on one of the components of the inhaler 10 that can be moved relative to another component of the inhaler 10, thereby indicating the position of the sleeve 42 within the casing 12. There are a number of examples of how this could be implemented. These are discussed below.

In the first example, a set of symbols 46 can be located on a surface of the threaded member 30. For example, the symbols 46 could be provided on one or more teeth 36 of the threaded member 30 or indeed on an outer surface of the handle 32 of the threaded portion 30. A corresponding window 48 or windows can be provided adjacent the teeth bearing the symbols 46 in the casing 12, so that the symbols 46 can be viewed through the casing 12. As one or more of the symbols 46 on the teeth 36 align with the windows in the casing 12, they become visible to a user holding the inhaler 10 and therefore give information to the user regarding the rotational position of the threaded member 30 within the casing 12. This in turn can provide information regarding the position of the sleeve 42 within the casing 12 and thereby provide information regarding the status of the medicament stored in the sleeve 42. Where the symbols 46 are provided on an outer surface of the handle 32, the windows described above can instead be provided in the flanged portion 20.

In another example, the symbols 46 described above can be provided on the sleeve 42 and a corresponding window 48 or windows can be provided in the casing 12 at an appropriate position along the length of the casing 12. As the sleeve 42 moves along the length of the casing 12, the symbols 46 would align with the window 48 or windows in the casing 12, thereby becoming visible to a user of the inhaler 10 as described above.

Accordingly, there has been described an inhaler for administering a predetermined dose of a medicament. The inhaler includes a screw-threaded mechanism which is operable to dispense the predetermined dose for inhalation. The inhaler can include a rotatable threaded member mounted within a casing. The inhaler can also include an internally threaded sleeve mounted on the rotatable threaded member. The sleeve can be moved linearly by rotation of the threaded member. Medicament can be provided in a tray of the sleeve, and can be aligned with a mouth piece of the inhaler for inhalation by the linear movement of the sleeve.

1. An inhaler for administering a predetermined dose of a medicament, the inhaler comprising:
a screw-threaded mechanism operable to dispense the predetermined dose for inhalation.

2. The inhaler of claim 1, further comprising a rotatable threaded member mounted within a casing.

3. The inhaler of claim 2, further comprising an internally threaded sleeve mounted on the rotatable threaded member, wherein the sleeve is linearly moveable by rotation of the rotatable threaded member.

4. The inhaler of claim 3, wherein the sleeve comprises a tray portion for storing one or more doses of a medicament to be inhaled.

5. The inhaler of claim 4, wherein the tray portion comprises a plurality of separate medicament storage sections, and wherein the internally threaded sleeve is linearly moveable by rotation of the threaded member for aligning one or more of the medicament storage sections with a mouth piece of the casing.

6. The inhaler of claim 4, wherein the tray portion comprises a cover for sealing a plurality of separate medicament storage sections prior to inhalation, and wherein the inhaler further comprises means for removing or breaking the cover as the sleeve moves linearly within the casing.

7. The inhaler of claim 4, wherein the tray portion is replaceable for replenishing the inhaler with medicament.

8. The inhaler of claim 2, wherein the casing is elongate and has a longitudinal axis, and wherein the rotatable threaded member is substantially coaxially mounted within the casing along the longitudinal axis.

9. The inhaler of claim 8, wherein the mouthpiece is positioned on a side of the elongate casing.

10. The inhaler of claim 2, wherein the casing comprises one or more air inlets to allow air to be drawn into the casing during inhalation of the predetermined dose of medicament.

11. The inhaler of claim 2, wherein the rotatable threaded member comprises a handle portion protruding from the casing for rotation by a user.

12. The inhaler of claim 2, wherein a thread of the threaded member has a pitch between about 1 and about 10 threads/cm.

13. The inhaler of claim 2, wherein a thread of the threaded member has a pitch between about 2 and about 5 threads/cm.

14. The inhaler of claim 3, wherein the inhaler comprises an indicator for indicating to a user information representative of the position of the sleeve within the casing, whereby the user can determine the status of the medicament within the inhaler.

15. The inhaler of claim 14, wherein the indicator comprises a window in the casing.

16. The inhaler of claim 15, wherein the indicator further comprises a set of symbols located on a surface of the rotatable threaded member, whereby rotation of the threaded member causes alignment of individual ones of the set of symbols with the window to indicate to the user the information representative of the position of the sleeve within the casing.

17. The inhaler of claim 16, wherein the symbols are located on a plurality of teeth of the rotatable threaded member.

18. The inhaler of claim 15, wherein the indicator further comprises a set of symbols located on a surface of the sleeve, whereby linear movement of the sleeve within the casing causes alignment of individual ones of the set of symbols with the window to indicate to the user the information representative of the position of the sleeve within the casing.

19. The inhaler of claim 2, further comprising a catch mechanism for locking the threaded member relative to the casing to inhibit inadvertent rotation of the threaded member within the casing.

20. The inhaler of claim 2, further comprising a rotating mechanism for providing stepwise rotation of the threaded member, whereby the threaded member can be rotated by a predetermined amount.

21. The inhaler of claim 2, wherein the threaded member is rotatable in a first direction within the casing.

22. The inhaler of claim 21, further comprising a ratchet mechanism for inhibiting rotation of the threaded member in a direction opposite to the first direction within the casing.

23. The inhaler of claim 2, further comprising a lock-out mechanism for preventing removal of the threaded member from the casing after the medicament in the inhaler has been exhausted.
